(19) United States
(12) Patent Application Publication
(10) Pub. No.: US 2010/0092954 A1
(43) Pub. Date: Apr. 15, 2010

(54) Method for Determining the Genetic Basis for Physiological Changes in Organisms

(76) Inventors: Bernhard Palsson, La Jolla, CA (US); Christopher D. Herring, Lebanon, NH (US)

Correspondence Address: DLA PIPER LLP (US), 4365 EXECUTIVE DRIVE, SUITE 1100, SAN DIEGO, CA, 92121-2133, US

(21) Appl. No.: 12/376,101

(22) PCT Filed: Aug. 1, 2007
(86) PCT No.: PCT/US07/17283

§ 371 Date: Dec. 8, 2009

Related U.S. Application Data
(60) Provisional application No. 60/835,368, filed on Aug. 2, 2006.

(51) Int. Cl. C12Q 1/68

(52) U.S. Cl. 435/6

(57) ABSTRACT

The present invention provides a method for discovering the basis of changes in the observable properties of organisms by subjecting an organism to selection. Individual organisms with observable differences will thus become more prevalent in a population during selection. The basis for the differences are then determined by identifying genetic differences among the individual organisms and the original organism, followed by evaluation of the effects of each genetic difference either alone or in combination by using site-directed mutagenesis followed by observation of the effects.

### BACKGROUND OF THE INVENTION

1. Field of the Invention

The invention relates generally to determining the genetic basis for physiological changes in organisms, and more specifically to use of experimental adaptation to generate strains of an organism having one or more changed phenotypes as compared to the wild type organism.

2. Background Information

All cellular behaviors involve the simultaneous function and integration of many interrelated genes, gene products and chemical reactions. Because of this interconnectivity, it is nearly impossible to a priori predict the effect of a change in a single gene or gene product in an organism, or the effect of a drug or an environmental factor, on the cellular behavior of an organism. The ability to accurately predict cellular behavior of an organism under different conditions would be extremely valuable in many areas of medicine and industry.

Genome sequencing technology provides the ability to determine the genomic sequence or information content of a genome. However, the meaning of this information is very difficult to interpret. For instance, while the location and sequence of a gene may be known, the function of that gene and how it affects the properties of the organism is often totally unknown. Thus, an understanding of how different changes in the genetic material of an organism lead to different observable properties of the organism is needed. This relationship is a fundamental topic of biology, and the development of methods that directly address this topic is vital to the progress of science.

Despite its importance to human health and industry, understanding the genotype:phenotype relationship is a difficult endeavor because of the complexity of living things. The underlying genetic basis of a bacterial property or characteristic can be difficult to determine because of the large number of genes and physiological systems that contribute to a specific property. For example, one strain of the bacterium, Escherichia coli, may cause a life-threatening infection while another may coexist peacefully in the human gut. This property, called pathogenesis, is the result of many underlying genes and physiological systems, such as genes for the production of toxic compounds and attachment to human cells. The complex genetic basis makes the understanding of pathogenesis slow and difficult.

Previous methods to determine the genetic basis of bacterial properties have focused on single genes or on groups of genes with known functions. Classical gene mapping allows a gene responsible for a phenotype to be located on a bacterial chromosome, but such methods fail when a phenotype is determined by more than one gene. Alternatively, a set of candidate genes can be sequenced to identify genetic differences that correlate with an observable property. A key factor in the success of this approach is the choice of which candidate genes to sequence. The choice is often dictated by the current knowledge of gene functions and by expectations based on current understanding of the organism. However, the functions of many genes that impact bacterial properties are not known. In addition, genes can have multiple functions or can impact widely diverse properties. Thus, a method to determine the genotype:phenotype relationship by sequencing only genes with known functions will often fail.

An effective method to determine the genetic basis of bacterial properties needs to correlate a given property to genetic differences in genes of unknown function, genes with no known relationship to that property, and functional elements outside the gene coding regions. This can be accomplished by examining all the genes and functional elements in intergenic regions present in an organism by complete genome sequencing. This approach has not been feasible, though, due to the large cost of sequencing a bacterial genome. A new type of sequencing called “resequencing,” based on highly parallel technologies such as microarrays or fluorescently labeled micro-beads, determines the sequence of a bacterial genome using a reference sequence that has already been completely determined. However, resequencing may have lower accuracy and requires that the organism to be resequenced be relatively similar to the reference organism.

Correlation of genome sequence differences to bacterial properties is not sufficient to establish the genotype:phenotype relationship in an organism because of the occurrence of false positives, which are typically sequence differences that correlate to a property, but occur by random chance and do not actually cause or affect the property. Use of resequencing may identify hundreds of sequence differences between two strains of bacteria, but only a few may be responsible for the difference in properties. Conversely, resequencing can be complicated by the occurrence of a false negative, which is the failure to detect a sequence difference that causes or affects the property.

The central dogma of molecular biology states that information flows from DNA to RNA to proteins. From there, the biochemical activity of a protein determines how the information contained in a gene affects the observable properties of an organism. Therefore, it is logical to try to understand the genotype:phenotype relationship by examining the biochemical activity of proteins. This is often accomplished by purifying the protein and by designing an assay to measure its biochemical activity in vitro. Unfortunately, proteins often have unexpected interactions with other proteins or other properties that are not easily mimicked in vitro. Moreover, it is difficult to extrapolate from biochemical activities to the complex properties of an organism.

The way that the observable properties of an organism are determined by the content of the genetic material is a complex topic and current tools to study them are generally insufficient. Thus there is a need for improved methods to unravel the complicated genotype:phenotype relationship within an organism by examining the biochemical activities of proteins in the context of the whole organism and taking advantage of the power of selection to establish the causality of sequence differences that are correlated to properties.

### SUMMARY OF THE INVENTION

The present invention provides a method for discovering the genetic basis for changes in the observable properties of organisms. The methods described herein include choosing a genetically-characterized organism and subjecting it to selection, where individual organisms with observable properties (i.e., phenotypes) will become more prevalent in a population. The basis of those differences are determined by identifying genetic differences between the organisms that were subjected to selection and the original organism, followed by evaluation of the effects of each genetic difference either alone or in combination by using site-directed mutagenesis followed by observation of the effects. In one embodiment, the organism is a microorganism such as a bacterium or single-celled fungus. For example, the organism may be E. coli or S. cerevisiae.

The methods include subjecting a first microorganism to a selected environment, wherein the microorganism is capable of genetic adaptation and performing selection over one or more generations to produce a second microorganism producing an observable phenotype, wherein the second microorganism contains one or more genetic changes as compared to the first microorganism. Thereafter, re-sequencing the genome or portion of the genome including the genetic change in the second microorganism. Then introducing the genetic change observed in the second microorganism into the first microorganism to produce a third microorganism, and determining that the same observable phenotype is present in the second and third microorganisms, thereby identifying the genetic basis for a phenotype. In one embodiment, the method further includes evaluating the observable phenotype of the third microorganism in comparison to the second microorganism. In another embodiment, the change is evaluated at a genetic level. In another embodiment, the change is evaluated at the level of gene products. In another embodiment, the method further includes performing genetic characterization prior to resequencing the genome, and may also including identifying one or more differences in the genetic characterization of the second microorganism as compared to the first microorganism. In another embodiment, the genetic change may be one or more mutations in one or more genes of the organism. In another embodiment, the genetic change may be one or more mutations in one or more regulatory regions of the genome.

In all aspects, the environment may be a batch culture, continuous culture, or a culture on a solid medium. In other aspects, the environment may be a single condition or multiple conditions. Such conditions include, but are not limited to an overabundance of one or more nutrients required for growth, a scarcity of one or more nutrients required for growth, an extended period of time (e.g. 4 to 180 days), and toxic substances (e.g., heavy metals, antibiotics and chlorinated compounds). The selection may be based on the ability of some individuals in a population to either grow faster or survive longer than others in the population

### BRIEF DESCRIPTION OF THE DRAWINGS

FIG. 1 is a graphical diagram showing the effects of mutations found in glycerol-adapted clones. For each mutant, the names of the mutated genes are given and in parentheses, the population from which the mutation was identified. The growth rate increase was calculated by dividing the growth rate increase of the mutant over WT by the increase of the endpoint clone over WT. Error bars indicate standard deviation.

FIG. 2 is a series of graphical diagrams showing Allele Frequency Estimation (AFE). The prevalence of mutations in each population was measured over the course of experimental evolution using hME genotyping and MassARRAY (Sequenom, San Diego, Calif.). The mutant alleles are denoted with symbols as indicated in the legend of each population. The average maximum error over all replicate measurements was 0.015 and thus too small to be meaningfully represented with error bars. The bottom left panel shows the growth rates of the evolving populations over time.

### DETAILED DESCRIPTION OF THE INVENTION

The present invention is based on methods that identify causality of mutations by introducing them back into an original strain of an organism, thereby evaluating the effects of the mutations. Thus, the present invention utilizes experimental evolution to generate strains of an organism having one or more changed phenotypes as compared to the original organism.

Before the present methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Comparative genomics has been almost entirely focused on genomic changes over long periods of time on the order of millions of years. A new microarray-based method of whole-genome resequencing called Comparative Genome Sequencing (CGS) now makes it cost efficient to monitor bacterial evolution comprehensively over short time periods as well. This capability is important because many microbial phenomena, such as the emergence of new pathogens and the acquisition of antibiotic resistance factors occur, over relatively short time scales. Experimental evolution of bacteria and viruses is a facile approach to study these topics, yet much remains unknown about genome plasticity over short evolutionary time scales.

It has been estimated that nearly 10% of the individuals in a Salmonella population carry large-scale genome rearrangements, and in a sub-optimal environment, selection can alter a population very rapidly. The 10-20 generations that occur in the process of growing a bacterial culture are sufficient to create a heterogeneous population, depending on the magnitude of the selective advantage of adaptive mutations.

Accordingly, the present invention provides a method for identifying the genetic basis for a phenotype in an organism. The method includes subjecting a first organism to a selected environment and performing selection over one or more generations to produce a second organism producing an observable phenotype, wherein the second organism contains one or more genetic changes as compared to the first organism. The genome or a portion of the genome large enough to include the genetic change in the second organism is then re-sequenced to identify the one or more genetic changes in the second organism. The genetic change(s) are then introduced into the first organism to produce a third microorganism. Finally, a determination that the same observable phenotype is present in the second and third organism indicates that the genetic basis for a phenotype has been identified. In one embodiment, the selection is performed over one or more generations. In another embodiment, the organism is a microorganism, such as a bacterium.

Thus, in one embodiment, the present invention provides a method to effectively determine the genetic basis of bacterial properties, while addressing both false positives and false negatives. The methods provide the ability to not only establish that a given sequence difference correlates with a property (i.e., phenotype), but also that the sequence difference causes or affects the property.

As used herein, the term “properties” refers to the observable characteristics of an organism, such as growth rate and the ability to utilize different media components. Such properties are often referred to as phenotypic properties or organism phenotype. As used herein, the term “phenotype” refers to the observable physical or biochemical characteristics (i.e., properties) of an organism, as determined by genetic makeup and environmental influences.

As used herein, the term “culturable organism” refers to any living organism that can be maintained and grown in a laboratory. It should be understood that organisms useful in the methods of the invention include, but are not limited to, any organism for which the gene or genome region of interest is experimentally unmodified prior to performing the steps of the invention. Accordingly, as used herein, the term “wild type,” when used in reference to an organism, refers to such experimentally unmodified organisms. However, “wild type organisms” may be distant descendants of an organism isolated from nature, and thus may already contain one or more sequence variants not normally found in nature. As used herein, the term “generation” refers to a descendant or offspring of an organism. It should be understood that each successive generation may contain one or more genetic changes as the organism adapts to a selected environment.

As used herein, the terms “selected environment,” “condition” or “conditions” refer to any external property that causes an organism to genetically adapt, evolve, change or mutate for survival. Exemplary “conditions” or “environments” include, but are not limited to, a particular medium, volume, vessel, temperature, mixing, aeration, gravity, electromagnetic field, cell density, pH, nutrients, phosphate source, nitrogen source, symbiosis with one or more organisms, and interaction with a single species of organism or multiple species of organisms (i.e., a mixed population). Also included as “conditions” or “environments” are substances that are toxic to the organism, such as heavy metals, antibiotics and chlorinated compounds. It should be understood that time may also be considered a “condition” since organisms are not static entities. Thus, a culture grown over an extended period of time (e.g., days, weeks, months, years) may produce different strains over the course of its genetic adaptation. An exemplary period of time is 4 to 180 days.

As used herein, the term “clone” refers to a single cell or population of cells that originated from a single cell. A clone is known to consist of cells with only one genotype or to have had a single genotype previously. The term “population” is intended to mean a group of individuals or cells. A “mixed population” therefore refers a group of cells from multiple species or to the collective genomes of naturally occurring organisms.

As used herein, the term “medium” or “media” refers to the chemical environment to which an organism is subjected or is provided access. The organism may either be immersed within the media or be within physical proximity thereto. Media are typically composed of water with other additional nutrients and/or chemicals that may contribute to the growth or maintenance of an organism. The ingredients may be purified chemicals (i.e., “defined” media) or complex, uncharacterized mixtures of chemicals such as extracts made from milk or blood. Standardized media are widely used in laboratories. Examples of media for the growth of bacteria include, but are not limited to, LB and M9 minimal medium. The term “minimal” when used in reference to media refers to media that support the growth of an organism, but are composed of only the simplest possible chemical compounds. For example, M9 minimal medium is composed of the following ingredients dissolved in water and sterilized: 48 mM Na₂HPO₄, 22 mM KH₂PO₄, 9 mM NaCl, 19 mM NH₄Cl, 2 mM MgSO₄, 0.1 mM CaCl₂, 0.2% carbon and energy source (e.g., glucose).

As used herein, the term “culture” refers to medium in a container or enclosure with at least one cell or individual of a viable organism, usually a medium in which that organism can grow. As used herein, the term “continuous culture” is intended to mean a liquid culture into which new medium is added at some rate equal to the rate at which medium is removed. Conversely, a “batch culture,” as used herein, is intended to mean a culture of a fixed size or volume to which new media is not added or removed.

As used herein, the term “model organism” refers to an organism that has been extensively characterized for the purpose of generalizing knowledge gained from it to other organisms. Exemplary model organisms include, but are not limited to, Escherichia coli, Saccharomyces cerevisiae, Drosophila melanogaster, Caenorhabditis elegans, Arabidopsis thaliana and Mus musculus.

As used herein, the term “physiological system” refers to a subset of the genetic, chemical and biological components of an organism, organized by the high-level function that they serve and their interactions with each other.

As used herein, the term “genetic basis” refers to the underlying genetic or genomic cause of a particular observation. Also included in the term is the most important reason for the occurrence of the observation.

As used herein, the term “genetic” refers to the heritable information encoded in the sequence of DNA nucleotides. As such, the term “genetic characterization” is intended to mean the sequencing, genotyping, comparison, mapping or other assay of the information encoded in DNA. The scope (e.g., extent, scale, etc.) of the genetic characterization is substantially genomic in scale so that a comprehensive assessment of all the genetic elements (known or unknown) can be simultaneously assessed. Substantially comprehensive evaluation ideally includes a full genome-scale re-sequencing of the organisms genome. In cases where full genomic sequencing is not possible, such as due to extensive sequence repeat regions, a comprehensive draft of the genome sequence can be used in the method described.

As used herein, the term “genetic material” refers to the DNA within an organism that is passed along from one generation to the next. Normally, genetic material refers to the genome of an organism. Extra-chromosomal, such as organelle or plasmid DNA, can also be a part of the ‘genetic material’ that determines organism properties. As used herein, “regulatory region,” when used in reference to a gene or genome, refers to a DNA sequence that controls gene expression. As used herein, a “gene product” refers to biochemical material, either RNA or protein, resulting from expression of a gene. Thus, a measurement of the amount of gene product is sometimes used to infer how active a gene is.

As used herein, the term “genetic change” or “genetic adaptation” refers to one or more mutations within the genome of an organism. As used herein, the term “mutation” refers to a difference in the sequence of DNA nucleotides of two related organisms, including substitutions, deletions, insertions and rearrangements, or motion of mobile genetic elements, for example. The term “introduction,” as used herein, refers to the putting of something such as a genetic change into something else, such as an organism. As such, the term “mutagenesis” is intended to mean the introduction of genetic change(s) into an organism.

As used herein, the term “selection” refers to an increase in the frequencies of different ‘types’ of individuals within a population by removal or enrichment of some types more so than others, either intentionally or spontaneously. The nature of a “type” can be defined by genetic or physical differences and a type may consist of one or many individuals. Archetypal examples of selection include, but are not limited to, (1) rifampicin selection in E. coli, in which all cells within a population are inhibited from growing on solid medium except those few individuals that contain mutations in the gene encoding the beta subunit of RNA polymerase that confer resistance to rifampicin, and (2) growth rate selection, in which individuals that grow and reproduce more quickly become more prevalent in a population. An important consideration in conducting selection is to determine what the “selection is for” or what is “being selected,” that is to say, the genetic or physical difference that is favorable or unfavorable in a particular environment. In example (1) the selection is for the ability to grow in the presence of rifampicin and thus, for mutations in RNA polymerase. In example (2), the selection is for a growth rate that is faster than other individuals in the population and that can be passed from a parent cell to its offspring.

Selection can be a powerful tool in bacterial genetics. It takes advantage of principles of evolution and the occurrence of spontaneous mutations to generate strains of bacteria with specific properties. In some ways, selection is like the proposition of a problem to a bacterium, where survival or increased prevalence depends on the solution that the bacterium can respond with. The genetic basis of the solution does not matter, just the relevant property of the bacterium. Therefore, selection is not biased to a particular set of genes and does not rely on current knowledge.

As used herein, the term “adaptation” refers to an increase in the capacity of an organism to survive and transmit its genetic information to reproductively viable offspring in a particular condition or set of conditions. Adaptation is often thought of as a process of “fine-tuning,” where multiple beneficial changes are made in a heuristic process of adjustment of the underlying gene elements. This reflects the complex nature of biological systems and must be accounted for in study of the genotype:phenotype relationship.

As used herein, the term “evaluation” is intended to mean observations or measurements of an observable phenotype of an organism. Evaluation typically includes analysis, interpretation and/or comparison with the phenotype of another organism. It should be understood that a phenotype may be evaluated at both the genetic level and at the level of gene products. Further, a phenotype may be evaluated in terms of the behavior of the organism within the environment and/or the behavior of individual molecules or groups of molecules within the organism. Such comparisons are useful in determining the detailed function of mutated products resulting from genetic adaptation.

As used herein, the term “re-sequencing” or “resequencing” refers to a technique that determines the sequence of a genome of an organism using a reference sequence that has already been completely determined. It should be understood that resequencing may be performed on both the entire genome of an organism or a portion of the genome large enough to include the genetic change of the organism as a result of selection.

As used herein, the term “reconstruction” refers to the creation of something as a copy of another, usually by assembling constituent parts. A living organism can be reconstructed by changing the genetic information of a second organism so that it matches the first organism. For example, the genetic material of bacterium ‘A’ may be found to differ from bacterium ‘B’ at four different nucleotides. Bacterium ‘A’ can be reconstructed from bacterium ‘B’ by changing the four differing nucleotides in bacterium ‘B’ so that they match bacterium ‘A.’

As used herein, the term “step-wise” is intended to mean in the fashion of a series of events, one following the other in time. As used herein, the term “simultaneous” is intended to mean happening at the same time.

Thus, in one aspect, the acquisition and fixation of mutations that convey a selective growth advantage is monitored by whole-genome resequencing of a specific bacterium adapted to a selected condition (e.g., growth medium supplemented with glycerol or lactase as the carbon and energy source). Proof that the observed spontaneous mutations are responsible for improved fitness is obtained using single, double and triple site-directed mutants that have growth rates matching those of the evolved strains. The success of this new genome-scale approach indicates that real time evolution studies will now be practical in a wide variety of contexts.

As such, the genetic investigation of experimental evolution can reveal mechanisms of evolution and novel connections between genotype and phenotype. Recently developed methods such as Comparative Genome Sequencing (CGS) now allow this investigation on a genome scale, yielding insightful results when gene expression data may be difficult to interpret. This study demonstrates the efficacy of genome resequencing, mutagenesis, and Allele Frequency Estimation (AFE) for the investigation of experimental evolution in bacteria. A full appreciation of the plasticity of genomes and the capacity of bacteria to rapidly adapt to new environments will emerge as genome scale technologies such as CGS are applied to study experimental evolution.

The following examples are provided to further illustrate the advantages and features of the present invention, but are not intended to limit the scope of the invention. While they are typical of those that might be used, other procedures, methodologies, or techniques known to those skilled in the art may alternatively be used.

### Example 1

### Genetic Basis of Growth Rate Adaptation of E. coli to Medium Containing Glycerol

This example demonstrates how an organism can be adapted to a defined medium and selected based on growth rate advantage. It also teaches the use of methods to perform genetic characterization through the use of currently available genome-scale DNA re-sequencing methods, and methods that can be used to introduce identified sequence changes to evaluate the differences in the observable (growth rate) properties of the particular organism.

Accordingly, the acquisition and fixation of mutations that convey a selective growth advantage was monitored by whole-genome resequencing of Escherichia coli adapted to a growth medium supplemented with glycerol or lactase as the carbon and energy source. Proof that the observed spontaneous mutations were responsible for improved fitness was obtained using single, double and triple site-directed mutants that had growth rates matching those of the evolved strains. The success of this new genome-scale approach indicates that real time evolution studies will now be practical in a wide variety of contexts.

Despite a complete pathway for glycerol catabolism, large variations in the growth rates of various strains have been noted. Growth of the sequenced strain MG1655 was observed to differ from computational predictions based on flux balance analysis. Upon extended logarithmic growth in glycerol minimal medium, the growth rate was observed to increase 150% in as few as 20 days (˜223 generations) and to reach the computationally predicted optimum.

Escherichia coli strain MG1655, was obtained from the American Type Culture Collection (#47076) and maintained as a laboratory stock. Genetic information about the organism was obtained from GenBank (accession #U00096). The fully defined minimal medium M9 containing 0.2% glycerol as the sole carbon and energy source was chosen as the conditions to which the organism was to be subjected.

The selection was based on growth rate during the logarithmic phase of growth. The selection was implemented by growing five independent populations in 250 ml of the medium in 500 ml Erlenmeyer flasks at 30° C. using a stir bar for aeration for 44 days. Every day, optical density (OD) measurements were made and cells were diluted into fresh medium, estimating the amount of inoculum to use such that the culture would not enter the stationary phase of growth. Samples were frozen periodically (i.e., at days 2, 4, 6, 8, 10, 15, 20, 25, 30, 35, 40 and 44) during adaptation based on growth rate selection for later analysis. Growth rate was monitored over time as an observable property. When changes in the observed property were no longer observed, the selection process was stopped. Individual clones were isolated from each of the five selected populations of organisms named GB, GC, GD, GE and G2 resulting from five different selection processes.

Genomic DNA was isolated from the selected organisms and hybridized to microarrays by Nimblegen Systems, Inc. using a Comparative Genome Sequencing (CGS) strategy (Manjunatha, et al., Identification of a nitroimidazo-oxazine-specific protein involved in PA-824 resistance in Mycobacterium tuberculosis. Proc Natl Acad Sci USA 103, 431-6 (2006)). A total of 95 putative Single Nucleotide Polymorphisms (SNPs) were reported by Nimblegen in the five clones. Each putative SNP was checked by PCR amplification and sequencing using MassARRAY SNP Discovery (Sequenom), and reaffirmed by classical Sanger sequencing if detected. Out of 95 reported SNPs, 17 were confirmed. Of the confirmed mutations, nine were resolved as sequence differences at three loci between the sequenced strain of MG1655 and the strain used to start the experimental evolution.

In addition to SNPs, Nimblegen provided the location of probes that showed a difference between the test and reference samples on mapping arrays yet showed inconclusive results on the sequencing arrays. From this data 15 regions were chosen for PCR amplification and Sanger sequencing, selecting regions where multiple nearby probes were implicated. This process led to the identification of three deletions. In addition, a probable duplication was identified in clone GC-1 of a 1,313 Kb region including the replication origin between two copies of the mobile genetic element IS2 (b3044 & b4273). MassARRAY SNP Discovery and Sanger sequencing candidate genes representing ˜7% of the genome showed two additional mutations that were not detected by the microarrays, including a 9 by duplication in glpK and a 28 by deletion of the 3′ end of pdxK and part of the downstream transcriptional terminator in clone G2-1. All mutations from this example are shown in Table 1.

TABLE 1
Validated mutations in clones isolated after 44 days of experimental evolution.

| | | | | Gene position | | |
|---|---|---|---|---|---|---|
| Clone | Gene | Product/Function | Mutation | nt | Region | Genome position |
| GB-1 | glpK | glycerol kinase | a → t | 218 | coding | 4115028 |
| | rpoC | RNA polymerase β′ | deletion | 3132 . . . 3158 | coding | 4186504 . . . 4186530 |
| | | | 27 bp | | | |
| GC-1 | glpK | glycerol kinase | g → t | 184 | coding | 4115062 |
| | n/a | all genes between insC-5 and insD-6 | duplication | n/a | n/a | ~3189209 . . . 4497523 |
| | | | 1313 kb¹ | | | |
| GD-1 | glpK | glycerol kinase | g → a | 816 | coding | 4114430 |
| | rpoB | RNA polymerase β | a → t | 1685 | coding | 4180952 |
| | murE | peptidoglycan biosynthesis | a → c | 8 | coding | 93173 |
| GE-1 | glpK | glycerol kinase | a → c | 113 | coding | 4115133 |
| | rpoC | RNA polymerase β′ | c → t | 2249 | coding | 4185621 |
| | dapF | lysine/peptidoglycan biosynthesis | c → a | 512 | coding | 3993293 |
| G2-1 | glpK | glycerol kinase | duplication | 705 | coding | 4114541 |
| | | | 9 bp | | | |
| | rph-pyrE | RNAse PH/pyrimindine synthesis | deletion | rph: | Coding + | 3813882 . . . 3813963 |
| | | | 82 bp | 610 . . . end | intergenic | |
| | pdxK-crr | pyridoxal kinase/enzyme IIa glucose | deletion | pdxK: | coding + | 2534400 . . . 2534427 |
| | | | 28 bp | 833 . . . end | intergenic | |

¹Evident in CGS mapping data; not independently validated.

To evaluate the contributions of individual mutations to increased growth rate in glycerol minimal medium, each mutation was introduced into the wild-type strain using a site-directed mutagenesis strategy, called gene gorging. This method introduces any desired mutation without direct selection, and therefore leaves no antibiotic resistance gene or other trace of having been used. Briefly, primers were designed 500-600 by on either side of each mutation, then the 18 by recognition sequence for I-SceI was appended to both primers. The mutations were amplified from the endpoint clones, then cloned into pCRBluntII-Topo (Invitrogen). The mutations were verified by Sanger sequencing, then the plasmids transformed into the WT strain along with pACBSR using transformation and storage solution (TSS) (Epicentre) according to the manufacturers instructions. Colonies were resuspended in RDM (Teknova) then lambda Red and I-SceI were induced with arabinose. After 7-9 h incubation at 37° C., the cells were diluted and plated on LB plates+25 μg/ml chloramphenicol and grown overnight at 37° C.

For each mutation, 31-190 colonies were PCR amplified using primers adjacent to those used for mutagenesis so that any remaining plasmid DNA would not amplify if present. The resulting PCR products were screened by various methods depending on the mutation. Large deletions were identified by gel electrophoresis, while most mutations required restriction digestion to distinguish from WT. In cases where no restriction difference could be used, PCR products were screened by hME Genotyping (Sequenom) or Sanger sequencing. Screened colonies were saved by patching on LB plates. After screening, 1 or 2 clones carrying the mutant allele as well as a WT clone to serve as control were struck on LB. Colonies were screened for loss of the mutagenesis plasmid pACBSR by patching on LB and LB+chloramphenicol plates. Plasmid-free clones could often be identified in this simple way, but when this failed, they were restruck on LB and screened again until a chloramphenicol sensitive clone was identified. The mutants and controls were grown overnight in LB, then stored at −80° C. after the addition of glycerol.

Growth rates were measured in conditions identical to those in which the strains were evolved. Frozen stocks were inoculated directly into 50-60 ml M9 minimal medium+0.2% glycerol, then stirred at 30° C. overnight in 250 ml Erlemeyer flasks. The cultures were stirred at 37° C. for 4-7 h following inoculation, then at 30° C. overnight. The cultures were then diluted into pre-warmed M9 glycerol medium such that the OD600 was near 0.02. A volume of 250 ml medium in a 500 ml Erlemeyer flask was stirred at 1200-1400 rpm using a 6.3 cm Teflon stir-bar in a 30° C. water bath. Samples were removed approximately every hour without interrupting the stirring and measured in a Thermo Spectronic BioMate3 spectrophotometer at 600 nm. Data were plotted in semi-log to evaluate the linearity of the readings and to identify the logarithmic phase of growth. Growth rates based on the natural logarithm were calculated with the SLOPE function of Microsoft Excel, using only those values less than 0.3. At least two measurements were made for each site-directed mutant.

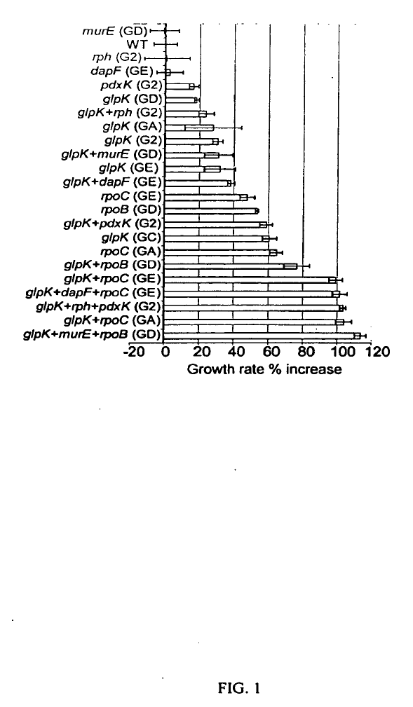

Growth rate measurements (FIG. 1) show that mutations in the two major subunits of RNA polymerase (rpoB and rpoC) conferred the largest change in growth rate, representing 48-65% of the total change. Mutations in glpK and pdxK also had significant effects. The 82 by deletion in rph probably relieves pyrimidine starvation documented in MG1655 caused by a frame-shift mutation in rph, but its effect on growth rate was undetectable. In addition to single-mutants, double- and triple-mutants were also made to reconstruct the clones isolated after experimental evolution. The growth rates of four such reconstruction strains matched the growth rates of the evolved clones from which they were reconstructions (GB-1, G2-1, GE-1 and GD-1). This indicates that all of the important mutations in these clones were able to be identified, and that there was no epigenetic component to the adaptation to glycerol minimal medium.

Sanger sequencing of ten likely candidate genes did not reveal any other mutations. It seems likely that the 1,313 Kbp genomic rearrangement in this strain either contributes to the growth rate or masks the presence of mutations in the duplicated region. Many of the genes implicated in this study (glpK, rpoBC, cyaA) are located in the duplicated region and may carry heterozygous mutations not detected by CGS or Sanger sequencing.

All clones had mutations in the gene for glycerol kinase (glpK), which catalyzes the first step in glycerol catabolism. This protein is subject to inhibition by fructose-1,6-bisphosphate (FBP) and phosphorylated enzyme IIA^{Glc}. Partially purified protein from cells expressing the mutant enzymes exhibited reaction rates 51-133% higher than WT, and five showed reduced inhibition by FBP (Table 2). Since all glycerol-adapted populations have mutations in glpK, rapid growth on glycerol can be partially attributed to altered kinetic and regulatory properties of glycerol kinase. Evidently the rate of glycerol phosphorylation catalyzed by GlpK is rate-limiting to the growth of WT E. coli MG 1655. The glycerol catabolic pathway generates the glycolytic intermediate dihydroxyacetone phosphate, which can be converted to methylglyoxal, a toxic metabolite when present at high concentrations. It is noteworthy that no mutations were detected in any genes relating to methylglyoxal metabolism.

TABLE 2
Biochemical measurements of mutant glycerol kinase

| | | | | Vmax μmol | |
|---|---|---|---|---|---|
| | Source | Amino acid | | glycerol/min/mg, | |
| Mutation | population | change | Location | (% increase over WT) | K_{I-FBP}(mM) |
| WT | | | | 15.8 | 0.5 |
| a218t | GA/GB | D73V | Interface of Subunit | 36 (128%) | >2 |
| | | | interaction | | |
| g184t | GC | V62L | Interface of Subunit | 29.8 (89%) | >2 |
| | | | interaction | | |
| g816a | GD | M272I | uncharacterized | 36.9 (134%) | >2 |
| a113c | GE | Q38P | EIIa^{Glc} interaction helix | 23.9 (51%) | 0.5 |
| g692a | G1 | G231D | FBP binding loop | 27.3 (73%) | >2 |
| Ins 9 bp | G2 | Insert KGG | FBP binding loop | 29.8 (89%) | >2 |

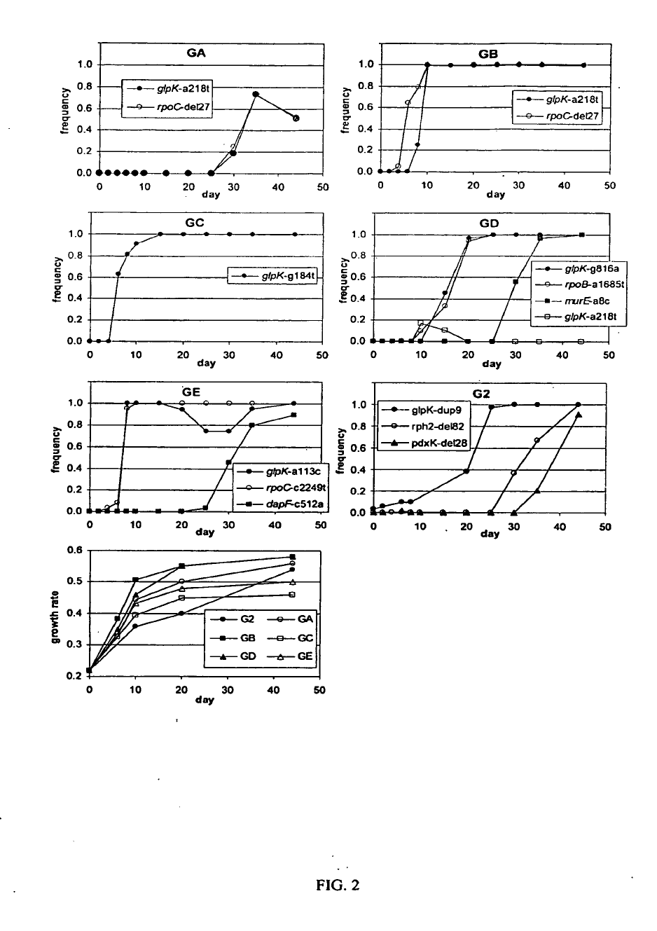

Mutations in genes involved in peptidoglycan biosynthesis (murE and dapF) were identified in two different clones. DapF produces the metabolite meso-2,6,-diaminopimelate, while MurE consumes it. It is unclear what advantage these mutations confer, since site-directed mutations in these genes had negligible effects. The only exception was the glpK+rpoB+murE triple-mutant, which grew faster than the glpK+rpoB double-mutant. There did not appear to be a relationship between murE or dapF mutations and glycerol metabolism. Since the mutations occurred after RNA polymerase mutations were fixed (FIG. 2), they might compensate for some detrimental side-effect of RNA polymerase mutations.

The gene pdxK encodes pyridoxine kinase, involved in vitamin B6 salvage. Since there is no direct connection between pdxK and glycerol metabolism, it is interesting to note that pdxK is adjacent to crr, encoding enzyme IIA^{Glc}, a regulator of glycerol kinase and a critical component in catabolite repression. The deletion at the 3′ end of pdxK results in deletion of part of a terminator between the convergently-transcribed genes pdxK and crr and might be able to attenuate enzyme IIA^{Glc} expression through some interesting mechanism, such as anti-sense inhibition.

The clone G1-1 carries a mutation in cyaA, encoding another key player in catabolite repression, adenylate cyclase. The 5 by deletion near the 3′ end of cyaA is likely to result in a truncated protein. It has been previously reported that a similarly truncated mutant of adenylate cyclase was insensitive to repression by glycerol-3-phosphate. It therefore appears that WT strain MG1655 grows slowly on glycerol because some critical genes are mis-expressed through the action of catabolite repression stimulated by glycerol-3-phosphate, and that the attenuating mutations in cyaA and pdxK/crr relieve this mis-expression.

Thus, much of the increase in growth rate seems to be achieved by a transcriptional adjustment. The clones GA-1, GB-1, GD-1, and GE-1 may accomplish the same effect through mutations in RNA polymerase (RNAP). It is possible that the RNAP mutations affect the level of catabolite repression by affecting RNAP interactions with catabolite activator protein CAP, but this explanation seems unlikely because CAP interacts with the a subunit of RNAP and the mutations are in the β and β′ subunits. Another hypothesis is that the RNAP mutations achieve a beneficial transcriptional adjustment by affecting some other regulatory aspect, such as transcriptional pausing or anti-termination. Few transcriptional commonalities were identified between the evolved populations in previous expression studies, so the target genes affected by RNAP and catabolite repression mutations are unknown. It seems likely though, that WT growth in glycerol minimal medium is limited by the mis-expression of multiple genes; otherwise, mutations in just one or two regulatory sequences would have been detected, rather than the transcriptional machinery.

The fixation of mutations identified in endpoint clones was monitored using Allele Frequency Estimation (AFE) (FIG. 2). Samples of the evolving populations were collected and frozen at regular intervals. Genomic DNAs from the revived samples were PCR amplified and oligonucleotides adjacent to the mutations were extended and quantitated using MS, validating and calibrating each assay with artificial DNA mixtures. This technique has a limit of detection of 2%. In general, the dynamic behavior followed the same pattern; mutations were undetectable in early time points then became fixed over 4-20 days. Mutations with large fitness effects (glpK or rpoBC mutations) were fixed first and more quickly relative to mutations with small effects (dapF, murE, and rph mutations), as expected. An exception was the pdxK mutation in population G2, which only occurred in the last two time points, consistent with the slow rise in growth rate of this population. Another notable exception to the usual pattern was population GA, for which the two endpoint mutations arose together late in the time course and did not reach fixation. The same two mutations were fixed in GB at day 10, suggesting that GB was probably contaminated into GA around day 20. The growth rate of GA was lower than GB at that time, allowing the introduced cells from GB to take over. The lack of fixation of the GA mutations may be a sign of a selective sweep at day 44 when the experiment was concluded.

In GB, the rpoC mutation became detectable 4 days before the glpK mutation, though both mutations became fixed simultaneously at day 10, suggesting that the addition of the glpK mutation allowed the rpoC mutants to overcome competitors. Similarly, the rpoB and glpK mutations in population GD appear to have been fixed simultaneously. The glpK and rpoC mutations in GE also appear to have been fixed simultaneously, though the glpK mutation later decreased unexpectedly. This decrease may have been due to the rise of a more fit rpoC/glpK combination, which was then outcompeted when the dapF mutation occurred. The low frequency of the glpK mutation in G2 between days 0-10 is likely artifactual, caused by primer dimers in that particular assay. Overall, AFE results are consistent with the magnitude of fitness effects observed with site-directed mutagenesis and provide detailed insight into population dynamics.

The goals of the current study were to determine the genetic basis of the adaptation of E. coli to glycerol minimal medium and to observe the dynamic behavior of the mutations identified. Six populations of E. coli that were cultured in continuous logarithmic growth in glycerol minimal medium were chosen. Gene expression studies of these populations were previously performed, but did not reveal the underlying basis of adaptation. The results of resequencing 4.4% of the genomes of these populations using mass spectrometry (MS) have been previously reported. In the current study, comprehensive resequencing of these strains has been performed, the effects of mutations both genetically and biochemically have been evaluated, and their occurrence within the populations have been monitored.

These results show the histories of the evolutionary “winners” in the populations, but do not show alternate genotypes that may have been important in the outcome of the experiment. In order to observe alternate clonal lineages and to assess the amount of variation present during the course of experimental evolution, candidate genes in individual clones isolated from the evolving populations were sequenced at day 15. For strains GA, GB, GC, GD and GE, regions of ˜500 bp surrounding each endpoint mutation were resequenced using MassARRAY SNP Discovery (Sequenom, San Diego, Calif.) to identify alternate alleles in important genes (Table 3). Four alternate glpK alleles in GD and GE were identified, but none in the others. Surprisingly, two of the alternate alleles (a218t and g217t) affected the same amino acid (Asp73) as the a218t mutation in clones GA-1/GB-1. A mutation in this amino acid was identified in a previous study 20 and was shown to decrease inhibition by FBP and the formation of inactive tetramers. For the a218t mutation in GD, cross-contamination can be ruled out because the accompanying rpoC mutation from GB was not present in GD colonies. AFE analysis shows the transient occurrence of this allele in GD (FIG. 2). In GE, the rpoC-c2249t allele present in the endpoint was observed in 4 colonies together with a glpK allele which was not present in the endpoint clone. This suggests that the rpoC-c2249t allele occurred before the glpK alleles, in agreement with AFE results, and supports the explanation of the temporary decrease in the frequency of glpK-a113c.

TABLE 3
Genotypes of individual colonies isolated
after 15 days of experimental evolution.

| | Number of | | |
|---|---|---|---|
| Population | colonies | Genotype | Outcome¹ |
| GA | 96 | none | |
| GB | 80 | glpK-a218t rpoC-del27 | endpoint |
| GB | 16 | failed reactions | |
| GC | 93 | glpK-g184t | endpoint |
| GC | 3 | failed reactions | |
| GD | 97 | glpK-g816a rpoB-a1685t | endpoint |
| GD | 6 | glpK-a172c | transient |
| GD | 4 | glpK-a218t | transient |
| GD | 3 | failed reactions | |
| GE | 60 | glpK-a113c rpoC-c2249t | endpoint |
| GE | 6 | glpK-g22t | transient |
| GE | 4 | glpK-g217t rpoC-c2249t | transient |
| GE | 26 | failed reactions | |

¹Whether the indicated mutations persisted (endpoint) or were lost (transient).

Colony genotyping results were consistent with AFE results in all respects except for the frequency of the rpoB mutation in GD, which was 88% in colonies and 33% in AFE. This quantitative discrepancy can be explained by colony picking bias for large colonies, as the AFE assay was calibrated with artificial control mixtures. The mutations from endpoint clone GA-1 were not present at day 15 in population GA, consistent with the idea that GB contaminated GA around day 20. Most colonies in population GA had no detectable genotype but probably carried mutations in genes that were not screened. Growth rates of individual clones were measured, but the variation in growth rates appeared to be less than the noise in growth rate measurements, making it impossible to correlate growth rates to individual genotypes.

Now that the key genes have been identified, the adaptation of E. coli to glycerol may prove to be an excellent model for the study of clonal interference and other evolutionary phenomena. The results provided herein demonstrate that dramatic changes in phenotype can be mediated by as few as two mutations, in concordance with results in S. cerevisiae, maize and the influenza virus. It was also shown how a bacterium can be maladapted to a particular growth environment despite a complete metabolic pathway for the substrate. The most influential mutations identified fall into two classes; those affecting a specific function (e.g., the rate-limiting enzyme GlpK) and those affecting global transcription patterns.

### Example 2

### Scope of Variation in the Genetic Basis of Adaptation to Medium Containing Glycerol

Biological systems are known for their redundancy properties. That is, the same observable biological properties and changes therein can result from many equivalent or close-to-equivalent changes in the genetic material in an organism. Accordingly, this example teaches how such variations can be determined through the further development of the discovery of the genetic basis through the implementation of the taught method as implemented in Example 1.

The procedures from Example 1 were repeated examining many independent populations from many different selections, and only characterizing a portion of the genetic material. As such, this example was undertaken following Example 1 and utilizing the information gained from Example 1 to make the process of discovery of ‘variation in the genetic basis’ for growth rate improvement efficient. That is to say, the results from Example 1 led to the discovery of the most likely genomic locations and the genetic elements where mutations leading to changes in observable properties are located. Thus, the procedure of Example 1 was repeated multiple times to determine the variation in the mutations that lead to increased growth rates under the conditions that the chosen organism was subjected to.

Escherichia coli strain MG1655, was obtained from the American Type Culture Collection (#47076) and maintained as a laboratory stock. Genetic information about the organism was obtained from GenBank (accession #U00096). The fully defined minimal medium M9 containing 0.2% glycerol as the sole carbon and energy source was chosen as the conditions to which the organism was to be subjected.

The selection was determined to be for increased growth rate during the logarithmic phase of growth. The selection was implemented by growing 36 independent populations in 250 ml of the medium in 500 ml Erlenmeyer flasks at 30° C. using a stir bar for aeration for 44 days. Every day, optical density (OD) measurements were made and cells were diluted into fresh medium, estimating the amount of inoculum to use such that the culture would not enter the stationary phase of growth. Samples were frozen periodically for later analysis. Individual clones were isolated from each of the 36 populations. Genomic DNA was isolated and then PCR amplified using primers designed to amplify the genes rpoB, rpoC and glpK. The PCR products were sequenced using Sanger sequencing. Differences in the nucleotide sequences between the original and glycerol-adapted clones were identified by aligning the sequence data using SeqMan sequence assembly software. Twenty nine of the clones contained mutations in glpK, two of the clones contained mutations in rpoB and 22 of the clones contained mutations in rpoC (Table 4).

TABLE 4
Mutations identified in 36 populations selected in glycerol minimal medium.

| | | | Sequence | | | Sequence | | | Sequence |
|---|---|---|---|---|---|---|---|---|---|
| Population | Gene 1 | Position | Change | Gene 2 | Position | Change | Gene 3 | Position | Change |
| eBOP41 | glpK | 197 | c > a | rpoC | 3611-3619 | deletion | | | |
| eBOP42 | glpK | 55 | g > a | rpoC | 3121-3144 | deletion | | | |
| eBOP43 | glpK | 285 | g > a | rpoC | 3611-3619 | deletion | | | |
| eBOP44 | glpK | 710 | g > t | rpoC | 3611-3619 | deletion | | | |
| eBOP45 | glpK | 288 | g > t | | | | | | |
| eBOP46 | glpK | 55 | g > t | rpoC | 3129-3155 | deletion | | | |
| eBOP47 | glpK | 218 | a > c | | | | | | |
| eBOP48 | glpK | 218 | a > c | | | | | | |
| eBOP49 | glpK | 218 | a > c | | | | rpoB | 1577 | c > t |
| eBOP51 | glpK | 218 | a > t | rpoC | 3137-3163 | deletion | | | |
| eBOP52 | glpK | 816 | g > t | rpoC | 3611-3619 | deletion | | | |
| eBOP53 | | | | rpoC | 3611-3619 | deletion | | | |
| eBOP54 | glpK | 218 | a > c | rpoC | 3611-3619 | deletion | | | |
| eBOP55 | glpK | 542 | g > t | rpoC | 3611-3619 | deletion | | | |
| eBOP56 | glpK | 816 | g > a | rpoC | 3611-3619 | deletion | | | |
| eBOP57 | glpK | 218 | a > c | rpoC | 3611-3619 | deletion | | | |
| eBOP58 | glpK | 518 | a > g | rpoC | 3611-3619 | deletion | | | |
| eBOP59 | | | | | | | | | |
| eBOP61 | glpK | 973 | a > c | rpoC | 3611-3619 | deletion | | | |
| eBOP62 | | | | rpoC | 3611-3619 | deletion | | | |
| eBOP63 | glpK | 218 | a > t | rpoC | 3611-3619 | deletion | | | |
| eBOP64 | glpK | 163 | g > t | rpoC | 3611-3619 | deletion | | | |
| eBOP65 | glpK | 697 | a > g | rpoC | 3611-3619 | deletion | | | |
| eBOP66 | glpK | 284 | g > a | rpoC | 3611-3619 | deletion | | | |
| eBOP67 | glpK | 700 | g > a | rpoC | 3611-3619 | deletion | | | |
| eBOP68 | glpK | 218 | a > c | rpoC | 3611-3619 | deletion | | | |
| eBOP69 | glpK | 218 | a > c | | | | rpoB | 1921 | g > a |
| eBOP81 | | | | rpoC | 3611-3619 | deletion | | | |
| eBOP82 | | | | | | | | | |
| eBOP83 | | | | rpoC | 3611-3619 | deletion | | | |
| eBOP84 | glpK | 816 | g > a | | | | | | |
| eBOP85 | | | | | | | | | |
| eBOP86 | | | | rpoC | 3611-3619 | deletion | | | |
| eBOP87 | glpK | 218 | a > c | rpoC | 3611-3619 | deletion | | | |
| eBOP88 | glpK | 218 | a > t | rpoC | 3137-3163 | deletion | | | |
| eBOP89 | glpK | 191-193 | duplication | rpoC | 3611-3619 | deletion | | | |

Accordingly, this example teaches how to efficiently implement the methods of the invention for the discovery of variation in the genetic basis of adaptation. It shows how information from genome-scale genetic characterization (Example 1) can be used to obtain additional data in a rapid and cost effective way by limiting the genetic characterization step to just a few important genes. The variation indicates which amino acids and structural features of the important proteins are involved in the beneficial effects that were selected. This knowledge can then be used for the manipulation of those proteins to accomplish a desired result (i.e., protein engineering) and may generate new leads for additional discovery.

### Example 3

### Effects of Genetic Differences on the Observable Properties of the Organism

In order to evaluate the mutations identified in Example 1 on the observable properties of the organism (i.e., growth rate), a variation on the site-directed mutagenesis approach was performed. Instead of introducing the mutations into the original wild type, the mutations were removed from the glycerol-adapted clones. In Example 1, the clone G1-1 was found to have a G692A mutation in glpK, and G2-1 was found to have a nine base-pair duplication at position 705 of glpK. For this example, the original wild type sequence of glpK was introduced into clones G1-1 and G2-1, generating the strains named BOP38 and BOP28, respectively. The growth rates of these two strains were measured and found to have decreased by 38% and 20% respectively, as a result of the removal of the mutation.

Thus, the removal of the genetic changes identified in the selected organism under the chosen condition with the reassessment of the observable properties also leads to the identification of the genetic basis for selection and the development of organism properties.

### Example 4

### Basis of the Adaptation of E. coli to Medium Containing Lactate

The use of different selection conditions may lead to the identification of genetic changes in the same genes or in different genes. Understanding the relationship between selection conditions and the genes in which mutations occur will help to define what physiological systems are involved in the interaction of an organism with different environments. This knowledge can then be used to manipulate the genes of that organism for a desired outcome.

In this example, the procedures from Example 1 were repeated, except that M9 minimal medium with 0.2% lactate as the sole carbon and energy source was chosen as the growth medium and the conditions for selection. A total of 93 putative SNPs were reported by Nimblegen in the five clones. Putative SNPs were checked by PCR amplification and Sanger sequencing. One of the confirmed mutations has been introduced into the original wild type clone.

The results in this example are informative identify the genes that are important for rapid growth in media containing lactate. However, when taken together with results from Example 1, it can be seen that mutations occur in genes directly related to the environment (e.g., glpK, ppsA) as well as mutations in genes affecting global regulatory systems (e.g., rpoB, relA). Current approaches to metabolic engineering of bacteria focus on genes directly related to a particular pathway, but the information generated in this example may lead to the discovery of mutations in global regulatory genes that are also useful for metabolic engineering objectives.

### Example 5

### Effects of Introduced Genetic Differences on the Observable Properties of an Organism Using Competition

Different combinations of mutations can also have the same effect on an organism. In Example 1, clone GB-1 had mutations in glpK and rpoC, whereas clone G2-1 had mutations in glpK, rph and pdxK, yet both clones show increased growth rates compared to the wild type parent strain. Thus, there are different ways in which growth rates can be increased. This means that selection does not always result in the same outcome, and there is some aspect of chance involved. In order to use selection for the manipulation of the properties of an organism, it is important to understand the role that chance plays as well as the factors influencing the outcome of a particular selection.

In Example 1 above, clones were obtained that were genetically different yet had growth rates that were indistinguishable using measurements based on optical density. Competition experiments are a more sensitive means to measure relative growth rates, and may be used to investigate mutations with small effects.

The glycerol-adapted clones generated in Example 1 were grown in M9 minimal medium containing 0.2% glycerol and then all possible pairs of clones were mixed in approximately equal proportions, then diluted into 250 ml of the same medium. The cultures were grown at 30° C. using a stir bar for aeration for 7 days. Every day, optical density (OD) measurements were made and cells were diluted into fresh medium, estimating the amount of inoculum to use such that the culture would not enter the stationary phase of growth. Samples were frozen periodically for later analysis.

Allelotyping, a method for the measurement of the frequencies of different mutations in a population, for which reagents and equipment are sold by Sequenom Inc. (San Diego, Calif.), was used to monitor the frequencies of mutations in the gene glpK in DNA extracted from the frozen samples. It was found that the mutations associated with one of the two mixed clones became more prevalent in the cultures over time. The clone GD-1 had the fastest relative growth rate in every case. In another experiment, site-directed mutants containing mutations in the genes murE, dapF and rph were grown in M9 minimal medium with 0.2% glycerol, then each of the three cultures was mixed in approximately equal proportions with a culture of wild type strain MG1655, then diluted into 250 ml of the same medium. The cultures were grown at 30° C. using a stir bar for aeration for 2 days. Both days, optical density (OD) measurements were made and cells were diluted into fresh medium, estimating the amount of inoculum to use such that the culture would not enter the stationary phase of growth. Samples were frozen for later analysis. Allelotyping was used to measure the frequencies of the mutations over time. The mutation in rph was found to confer a growth rate advantage of approximately 10% over wild type, whereas the mutations in murE and dapF conferred no advantage.

Accordingly, this example teaches how competition experiments can be used as a way to compare alternate outcomes to a given selection. It was discovered that the alternate outcomes do not have equal growth rates, and further discovered which combination of mutations results in the highest growth rate. Competition was also used to determine the relative growth rates of strains that could not be distinguished otherwise.

### Example 6

### Basis of Adaptation of E. coli Strains Containing a Genetic Modification

In Examples 1 and 4, it was shown how different media conditions affect the results of a selection experiment. It is also useful to vary the organism that is used. If an organism is chosen that is very different from the first organism, it may be hard to attribute different outcomes to particular differences between the organisms. Therefore, it is often preferable to choose an organism that differs from the first organism in only one gene. Such an organism can be made by genetic modification of the original organism, which can include the removal of one gene or the addition of a gene that is not already present.

The procedures from Example 1 were repeated in this Example, except that instead of the wild type strain MG1655, a site-directed mutant deleted for the gene pgi was used. This gene encodes glucosephosphate isomerase, the second step of glycolosis. M9 minimal medium with 0.2% glucose was chosen as the growth medium. The glucose-adapted strains have been stored frozen for later analysis. DNA will be extracted from a few independent clones then subjected to CGS as in Example 1. Mutations will be validated using Sanger sequencing, then introduced into the wild type strain and the effects on growth rate will be evaluated.

Accordingly, this example teaches how to discover how the presence or absence of a given gene affects the results of selection. In the case of a pgi-knockout, E. coli surmounts the loss of a gene in central metabolism by routing metabolic flux through alternate routes. This will lead to the discovery of genes and physiological systems that are important for the diversion of flux through metabolism, a key element in efforts to manipulate organisms for commercial ends.

### Example 7

### Basis of Adaptation of E. coli Strains Containing Multiple Genetic Modifications

Variation of the organism that will be adapted to a given set of conditions need not differ from a reference organism in just one gene. Multiple genes can be genetically modified to discover additional properties of an organism.

In this example, strains were generated for the commercial production of lactate by deleting two genes from their genetic material and then adapting them to minimal medium with glucose as the sole carbon and energy source. It was found that after adaptation to glucose minimal medium, the growth medium contained an increased titer of lactate relative to the wild type strain. Understanding the basis of change in the properties of the organism will be of commercial benefit because it will allow the generation of strains that produce lactate or some other product more efficiently.

The procedures from Example 1 were repeated in this Example, except that instead of the wild type strain MG1655, a site-directed mutant deleted for the genes pta and adhE was used. These genes encode the enzymes phosphate acetyltransferase and alcohol dehydrogenase, respectively. M9 minimal medium with 0.2% glucose was chosen as the growth medium. The glucose-adapted strains have been stored frozen for later analysis. DNA will be extracted from a few independent clones then subjected to CGS as in Example 1. Mutations will be validated using Sanger sequencing, then introduced into the wild type strain and the effects on growth rate will be evaluated.

### Example 8

### Basis of Adaptation of E. coli to Multiple Selections

In Example 1, a single selection was used—increased growth rate in glycerol minimal medium. It is also possible to use multiple selections, for the purpose of scientific investigation or for the generation of a useful strain. An example of multiple selections chosen for their scientific interest are combined selections for increased growth rate and for survival and rapid recovery from stationary phase. An example of multiple selections chosen for the generation of a useful strain are combined selections for increased growth rate and for increased production of some product.

### Example 9

### Basis of Adaptation of E. coli for Different Periods of Time

Allelotyping results from populations generated in Example 1 showed that the various mutations appeared at different times. By adapting an organism for different amounts of time, it may be possible to intentionally generate strains with different mutations, similar to the task of site-directed mutagenesis. For example, it was found that mutations in glpK and rpoB or rpoC occurred very early in the time course of adaptation. By choosing clones from frozen stocks early in the course of the selection procedure, it is possible to obtain clones containing mutations only in glpK, rpoB or rpoC.

As shown in FIG. 2, the prevalence of mutations in each population was measured over the course of experimental evolution using hME genotyping and MassARRAY (Sequenom). The mutant alleles are denoted with symbols as indicated in the legend of each population. The average maximum error over all replicate measurements was 0.015 and thus too small to be meaningfully represented with error bars. The bottom left panel shows the growth rates of the evolving populations over time.

### Example 10

### Basis of Adaptation of E. coli to Growth in Continuous Culture

This example is essentially a repeat of example 1, except that instead of growing the organism in a series of batch cultures, the organism will be grown in a continuous culture.

Although the invention has been described with reference to the above example, it will be understood that modifications and variations are encompassed within the spirit and scope of the invention. Accordingly, the invention is limited only by the following claims.

What is claimed is:

1. A method for identifying the genetic basis for a phenotype in a microorganism comprising:
(a) subjecting a first microorganism to a selected environment, wherein the microorganism is capable of genetic adaptation;
(b) performing selection over one or more generations to produce a second microorganism displaying an observable phenotype, wherein the second microorganism contains one or more genetic changes as compared to the first microorganism;
(c) re-sequencing the genome or portion of the genome including to the genetic change in the second microorganism;
(d) introducing the genetic change into the first microorganism to produce a third microorganism; and
(e) determining that the same observable phenotype is displayed by the second microorganism and the third microorganism, thereby identifying the genetic basis for a phenotype.

2. The method of claim 1, further comprising evaluating the observable phenotype of the third microorganism in comparison to the second microorganism.

3. The method of claim 2, wherein the phenotype is evaluated at a genetic level.

4. The method of claim 2, wherein the phenotype is evaluated at the level of gene products.

5. The method of claim 1, wherein the genetic change is one or more mutations in one or more genes of the microorganism.

6. The method of claim 1, wherein the genetic change is one or more mutations in one or more regulatory regions of the genome.

7. The method of claim 1, wherein the genetic change is one or more mutations in the genome.

8. The method of claim 1, further comprising performing genetic characterization prior to re-sequencing the genome.

9. The method of claim 8, further comprising identifying one or more differences in the genetic characterization of the second microorganism as compared to the first microorganism.

10. The method of claim 1, wherein the microorganism is a bacterium.

11. The method of claim 10, wherein the microorganism is E. coli.

12. The method of claim 1, wherein the microorganism is a single-celled fungus.

13. The method of claim 12, wherein the microorganism is Saccharomyces cerevisiae.

14. The method of claim 1, wherein the environment is a batch culture or a series of batch cultures.

15. The method of claim 1, wherein the environment is a continuous culture or a series of continuous cultures.

16. The method of claim 1, wherein the environment is a culture on solid medium or a series of cultures on solid medium.

17. The method of claim 1, wherein the environment is a single condition.

18. The method of claim 1, wherein the environment is multiple conditions.

19. The method of claim 1, wherein the environment is a culture of a single species.

20. The method of claim 1, wherein the environment is a culture of multiple species.

21. The method of claim 1, wherein the environment is a series of dilutions into which a fraction of the first organism is transferred.

22. The method of claim 17, wherein the condition is an overabundance of one or more nutrients required for growth.

23. The method of claim 17, wherein the condition is a scarcity of one or more nutrients required for growth.

24. The method of claim 17, wherein the condition is an extended period of time.

25. The method of claim 24, wherein the extended period of time is about 4 to 180 days.

26. The method of claim 17, wherein the condition includes a toxic substance.

27. The method of claim 26, wherein the toxic substance is a heavy metal, antibiotic or a chlorinated compound.
